(19) United States
(12) Patent Application Publication
(10) Pub. No.: US 2003/0082550 A1
(43) Pub. Date: May 1, 2003

(54) Mutations of the cyclooxygenase-2 gene

(76) Inventors: Hans-Ulrich Thomann, Lexington, MA (US); Kristen Wall Diamond, Duxbury, MA (US); Michael G. FitzGerald, Waltham, MA (US)

Correspondence Address: BURNS DOANE SWECKER & MATHIS LLP, PO BOX 1404, ALEXANDRIA, VA, 22313-1404, US

(21) Appl. No.: 09/949,293

(22) Filed: Sep. 7, 2001

Related U.S. Application Data
(60) Provisional application No. 60/231,250, filed on Sep. 8, 2000.

(51) Int. Cl. C12Q 1/68; C12N 9/02; C07H 21/04

(52) U.S. Cl. 435/6; 435/189; 536/23.2

(57) ABSTRACT

This invention relates to isolated nucleic acids comprising single nucleotide polymorphisms of the human COX-2 gene and the proteins encoded by these nucleotides. The invention also relates to pharmacogenomics. The invention provides methods of using the polymorphisms in diagnostics and therapeutics.

### RELATED APPLICATIONS

This invention claims priority to U.S. Provisional Application No.: 60/231,250, filed Sep. 8, 2000, the contents of which are herby incorporated by reference.

### FIELD

This invention relates to the field of molecular biology and genomics. The invention also relates to pharmacogenomics. The invention provides polymorphisms of the COX-2 gene and the methods of using them in diagnostics and therapeutics.

### BACKGROUND

Cyclooxygenase (COX) is the key enzyme involved in the conversion of arachidonic acid to prostaglandins (PGs). PGs are involved in homeostatic functions as well as inflammatory responses (Dubois et al., FASEB, 12:1063-1073 (1998)). Some of the homeostatic or “house keeping” functions of PGs include blood clotting, ovulation, initiation of labor, bone metabolism, nerve growth and development, wound healing, kidney function, blood vessel tone and immune responses. In addition to its housekeeping functions, COX is associated with various diseases or sicknesses including but not limited to fever, arthritis including rheumatoid arthritis, Alzheimer's disease, seizures, bone conditions including osteoarthritis and other various inflammatory conditions. COX has also been shown to be involved in cancer and in particular, breast cancer (Harris et al., Cancer Research 60:2101-03 (2000)).

Two isoforms have been identified, cyclooxegenase 1 (COX-1) and cyclooxegenase 2 (COX-2). COX-1 is known to be present in most tissues and is constitutively expressed. COX-2, however, is primarily an inflammatory, inducible enzyme.

Most therapeutics available to treat inflammation include non-steroidal anti-inflammatory drugs (NSAIDs) which inhibit COX-1 and COX-2 activity. While NSAIDs are effective for various inflammatory conditions, 25% of individuals experience some type of side effect and 5% develop serious health consequences rom these treatments. These side effects frequently result from the inhibition of COX activity with regard to its homeostatic functions. Recently, additional therapeutics have been introduced to specifically inhibit COX-2 activity including but not limited to, meloxicam, nimesulide, etodalac, nabumetone, celecoxib, Rofecoxib, Dup 697 (Hawkey, LANCET, 353: 307-314 (1999)).

The side effects of some to the NSAIDs can also be attributed to the differences between individuals at the nucleotide or gene level. Thus, more information regarding the COX-2 gene and its inherent mutations is needed to develop better therapeutic drugs.

### SUMMARY

This invention relates polymorphisms of the COX-2 gene described in Table 3 and FIGS. 2A-2E. In one embodiment the invention relates to a fragment containing 15 continuous nucleotide bases which can be RNA or DNA and their complements. The fragment can be single or double stranded. Antisense oligonucleotides are also related to this invention as a therapeutic approach for inhibition of gene expression.

Another embodiment includes a probe containing a polymorphism of Table 3. In yet another embodiment the invention provides an allele specific oligonucleotide. The invention also provides a kit to identify individuals containing a COX-2 polymorphism.

The invention also provides a polypeptide comprising at least 5 consecutive amino acid bases encoded by the nucleic acids of Table 3.

In another embodiment the invention relates to antibodies that can be raised against all or a part of these amino acid sequences for specific diagnostic and therapeutic methods requiring such antibodies. These antibodies can be polyclonal, monoclonal, or antibody fragments.

Another embodiment includes a method of detecting COX-2 polymorphisms by restriction-fragment-length-polymorphism detection based on allele-specific restriction-endonuclease cleavage, hybridization with allele-specific oligonucleotide probes, oligonucleotide arrays, allele-specific PCR, mismatch-repair detection (MRD), denaturing-gradient gel electrophoresis (DGGE), single-strand-conformation-polymorphism detection, RNAase cleavage at mismatched base-pairs, chemical or cleavage of heteroduplex DNA, methods based on allele specific primer extension, genetic bit analysis (GBA, the oligonucleotide-ligation assay (OLA), the allele-specific ligation chain reaction (LCR) (Barrany Proc. Natl. Acad. Sci. U.S.A. 88:189-1 93 (1991)), gap, radioactive and/or fluorescent DNA sequencing using standard procedures well known in the art, and peptide nucleic acid (PNA) assays.

Another embodiment of this invention relates to pharmagenomics which includes a method of predicting a clinical response to a therapeutic compound, or for determining the therapeutic dose of a compound in the treatment of a COX-2 mediated disease.

The invention also relates to a method of assessing the predisposition of an individual to diseases mediated by COX-2. In another embodiment the invention includes a method of treating a human in need of COX-2 drug in which the method includes: (i) diagnosis of a single nucleotide polymorphism in the COX-2 gene in the human, which diagnosis comprises determining the sequence of the nucleic acid at one or more positions in Table 3, (ii) determining the status of the human by reference to polymorphism in the COX-2 gene; and (iii) administering an effective amount of a COX-2 drug.

And yet another embodiment includes a computer readable medium comprising at least one nucleic acid sequence of Table 3.

### BRIEF DESCRIPTION OF FIGURES

FIG. 1: Schematic representation of human cyclooxygenase-2 gene regions amplified using oligonucleotide primers

FIGS. 2A-2E:COX-2 Gene Sequence in GenBank Format (direct Genbank submission Nov. 23, 1999 by G. Coville at the Sanger Center, AC number AL033533). All polymorphisms are relative to this particular COX-2 sequence. Important regulatory elements, such as signal boxes or protein binding domains (Hla and Neilson, 1992) are underlined and annotated. Promoter modules identified with the software GEMS Launcher (Genomatix, Munich/Germany) are boxed and annotated. The ATG start codon is located at position 2377. Note, that COX-2 in mouse undergoes a post translational modification as the signal peptide, which is important for membrane translocation of the protein and which is represented by the first 17 amino acids encoded, is cleaved off (Kurumbail et al. 1996). The corresponding 51 nucleotides in the human gene are shown in underlined italics. All single nucleotide polymorphisms are shown in bold/underlined and are annotated according to their observed base change.

FIG. 3: Crystal structure of cyclooxygenase-2. The amino acids in the human COX-2 protein which are impacted by SNPs are visualized by superimposing them on the X-ray crystal structure of mouse COX-2, Protein Data Bank (H. M.Berman, J.Westbrook, Z.Feng, G.Gilliland, T. N.Bhat, H.Weissig, I. N.Shindyalov, P. E.Bourne, 2000.The Protein Data Bank. Nucleic Acids Research 28, 235-242) code 4cox (R. G.Kurumbail, A. M.Stevens, J. K.Gierse, J. McDonald, R. A.Stegeman, J. Y.Pak, D.Gildehaus, J. M.Miyashiro, T. D.Penning, KSeibert, P. C.Isakson, W. C.Stallings, 1996. Nature 384:644-648), using the using the molecular modeling software MidasPlus (T. E.Ferrin. C. C.Huang, L. E. Jarvis and R. Langdridge, 1998. J. Mol. Graphics 6:2-12).

FIGS. 4A-4B: BlastAlign of mammalian COX-2 proteins. Shown are blastalign (CLUSTAL W 1.8) results of COX-2 proteins from mouse, rat, guinea pig, rabbit, horse, sheep and human. Amino acids conserved between both proteins and all species are depicted by an asterisk. Amino acids which are conservative changes between species and proteins are marked with a semicolon. The amino acid changes caused by SNPs in the human COX-2 gene are boxed in the human sequence.

### DETAILED DESCRIPTION

For purposes of present invention the following terms are defined below:

“COX-2 drug” or “COX-2 inhibitor” includes agents, therapies, methods and pharmaceuticals which modulate the activity of COX-2 gene and its encoded protein. One example of such agent is Celecoxib which inhibits COX-2 enzyme. A COX-2 drug can be specific for COX-2 modulation or it can also include agents which may or may not modulate COX-1 gene and its encoded (or expressed) protein.

“Gene” refers to a DNA sequence that encodes through its template or messenger RNA a sequence of amino acids characteristic of a specific peptide. The term “gene” includes intervening, non-coding regions, as well as regulatory regions, and can include 5′ and 3′ ends.

The gene sequences of the present invention can be derived from a variety of sources including DNA, cDNA, synthetic DNA, synthetic RNA or combinations thereof. Such sequences may comprise genomic DNA which may or may not include naturally-occurring introns. Moreover, such genomic DNA may be obtained in association with promoter regions or poly (A) sequences. The sequences, genomic DNA or cDNA can be obtained in any of several ways. Genomic DNA can be extracted and purified from suitable cells by means well known in the art. Alternatively, mRNA can be isolated from a cell and used to produce cDNA by reverse transcription or other means. The term further includes variants resulting from alternative splice sites.

Nucleic acids referred to herein as “isolated” are nucleic acids separated away from the nucleic acids of the genomic DNA or cellular RNA of their source of origin (e.g., as it exists in cells or in a mixture of nucleic acids such as a library), and may have undergone further processing. “Isolated”, as used herein, refers to nucleic or amino acid sequences that are at least 60% free, prefereably 75% free, and most preferably 90% free from other components with which they are naturally associated. “Isolated” nucleic acids (polynucleotides) include nucleic acids obtained by methods described herein, similar methods or other suitable methods, including essentially pure nucleic acids, nucleic acids produced by chemical synthesis, by combinations of biological and chemical methods, and recombinant nucleic acids which are isolated

“Oligonucleotide” refers to a single stranded nucleic acid ranging in length from 2 to 60 bases. Oligonucleotides are often synthetic but can also be produced from naturally occurring polynucleotides. A probe is an oligonucleotide capable of binding to a target nucleic acid of complementary sequence through one or more types of chemical bonds, usually through complementary pairing via hydrogen bond formation. Oligonucleotides probes are often 5 to 60 bases and in specific embodiments may be between 10 and 40, or 15 and 30 bases long. An oligonucleotide probe may include natural (i.e. A, G, C or T) or modified bases (7-deazaguanosine, inosine, etc.). In addition, the bases may be joined by a linkage other than a phosphodiester bond, such as a phosphoramidite linkage or a phosphorothioate linkage, or they may be a peptide nucleic acids in which the constituent bases are joined by peptide bonds rather than by phosphodiester bonds, so long as it does not interfere with hybridization.

“Pharmacogenomics” or “pharmacogenetics” is the approach whereby a particular group of pharmaceutical agents are chosen to treat or diagnose disorders of an individual and/or class of individuals based on the polymorphisms of that individual or class. Pharmacogenomics or pharmacogenetics can also be used in the pharmaceutical research to assist in the drug selection process.

“Polymorphism” refers to the occurrence of two or more genetically or artifically determined alternative sequences or alleles in a population.

As used herein, the term “primer” refers to a single-stranded oligonucleotide which acts as a point of initiation of template-directed DNA synthesis under appropriate conditions (e.g., in the presence of four different nucleoside triphosphates and a polymerization agent, such as DNA polymerase, RNA polymerase or reverse transcriptase) in an appropriate buffer and at a suitable temperature. The appropriate length of a primer depends on the intended use of the primer, but typically ranges from 10 to 20 nucleotides or 15 to 30 nucleotides. Short primer molecules generally require cooler temperatures to form sufficiently stable hybrid complexes with the template. A primer need not be perfectly complementary to the exact sequence of the template, but should be sufficiently complementary to hybridize with it. The term “primer site” refers to the sequence of the target DNA to which a primer hybridizes. The term “primer pair” refers to a set of primers including a 5′ (upstream) primer that hybridizes with the 5′ end of the DNA sequence to be amplified and a 3′ (downstream) primer that hybridizes with the complement of the 3′ end of the sequence to be amplified.

“Reference sequence” is the nucleotide sequence of the COX 2 gene and the corresponding amino acid sequence of the COX-2 protein as described by Genbank submission Nov.23, 1999 by G. Coville at the Sanger Center (AC number AL033533).

“Single nucleotide polymorphism” or “SNP” occurs at a polymorphic site occupied by a single nucleotide which is the site of variation between allelic sequences.

The invention relates to SNPs of the COX-2 gene. The methodology described in Example 1 is not meant to be limiting. The detection of polymorphisms in specific DNA sequences, can be accomplished by a variety of methods including, but not limited to, restriction-fragment-length-polymorphism detection based on allele-specific restriction-endonuclease cleavage Kan and Dozy Lancet ii:910-912 (1978)), hybridization with allele-specific oligonucleotide probes (Wallace et al. Nucl Acids Res. 6:3543-3557 (1978)), including immobilized oligonucleotides (Saiki et al. Proc. Natl. Acad. Sd. USA 86:6230-6234 (1969)) or oligonucleotide arrays (Maskos and Southern Nucl Acids Res21:2269-2270 (1993)), allele-specific PCR Newton et al. Nucl Acids Res 17:2503-2516 (1989)), mismatch-repair detection (MRD) (Faham and Cox Genome Res 5:474-482 (1995)), binding of MutS protein (Wagner et al. Nucl Acids Res 23:3944-3948 (1995), denaturing-gradient gel electrophoresis (DGGE) (Fisher and Lerman et al. Proc. Natl. Acad. Sci. USA. 80:1579-1583 (1983)), single-strand-conformation-polymorphism detection (Orita et al. Genomics 5:874-879 (1983)), RNAase cleavage at mismatched base-pairs (Myers et al. Science 230:1242 (1985)), chemical (Cotton et al. Proc. Natl. Acad. Sci. U.S.A, 8Z4397-4401(1988)) or enzymatic (Youil et al. Proc. Natl. Acad. Sci. U.S.A. 92:87-91(1995)) cleavage of heteroduplex DNA, methods based on allele specific primer extension (Syvanen et al. Genomics 8:684-692 (1990)), genetic bit analysis (GBA) Nikiforov et al. Nucl Acids 22:4167-4175 (1994)), the oligonucleotide-ligation assay (OLA) (Landegren et al. Science 241:1077 (1988)), the allele-specific ligation chain reaction (LCR) (Barrany Proc. Natl. Acad. Sci. U.S.A. 88:189-1 93 (1991)), gap-LCR (Abravaya et al. Nud Acids Res 23:675-682 (1995)), radioactive and/or fluorescent DNA sequencing using standard procedures well known in the art, and peptide nucleic acid (PNA) assays (Orum et al., Nuci. Acids Res, 21:5332-5356(1993).

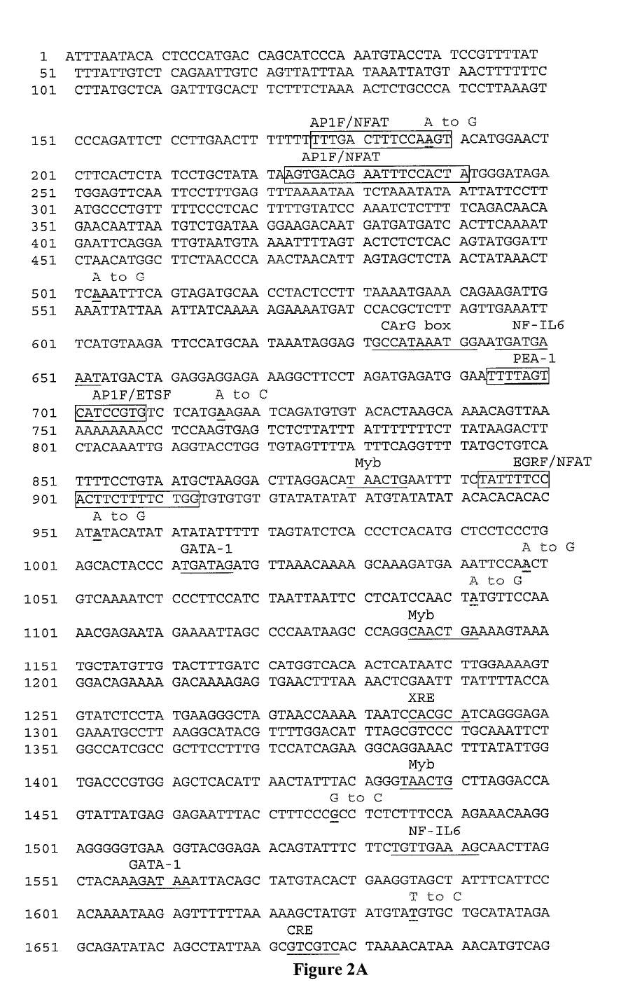

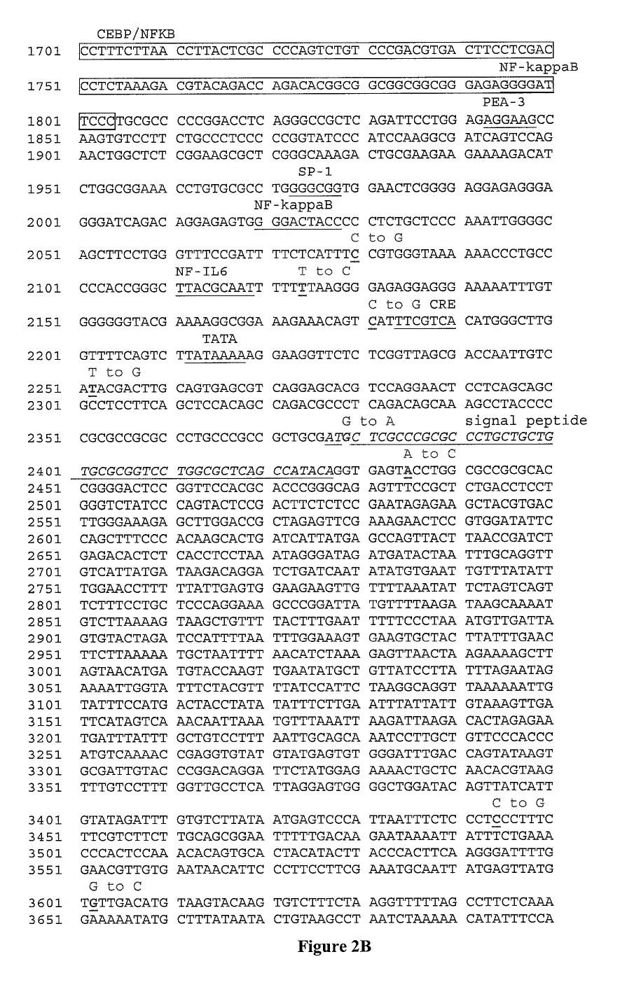

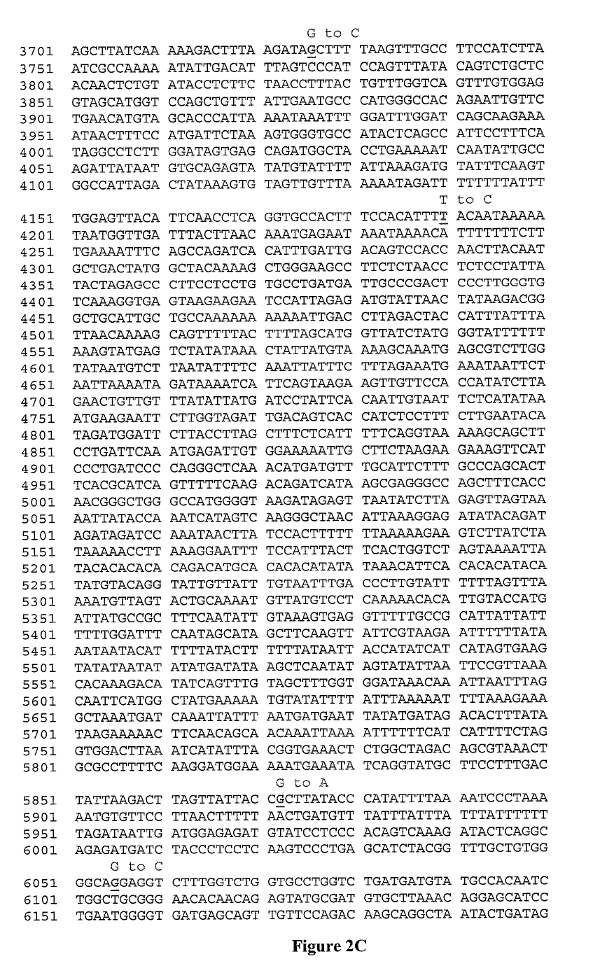

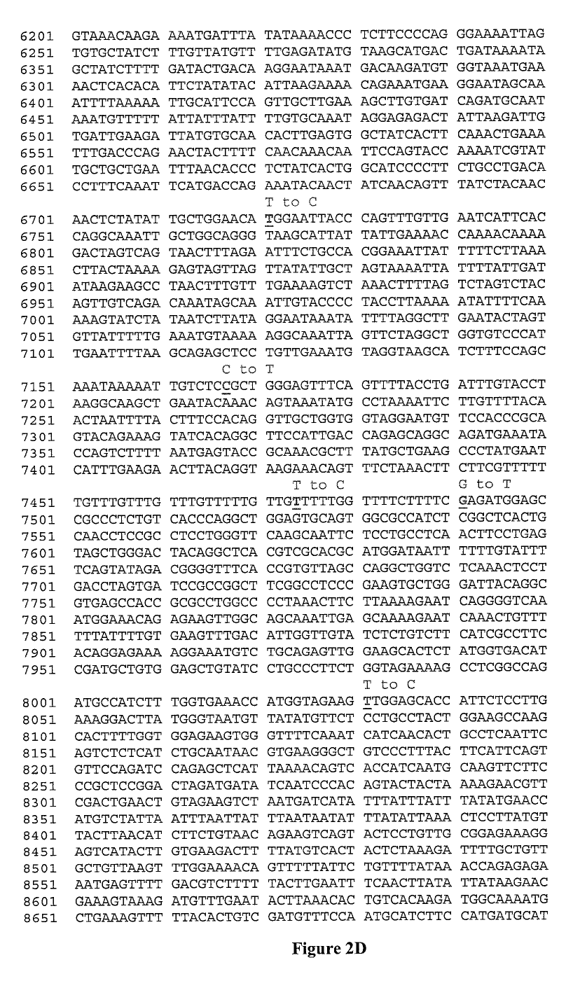

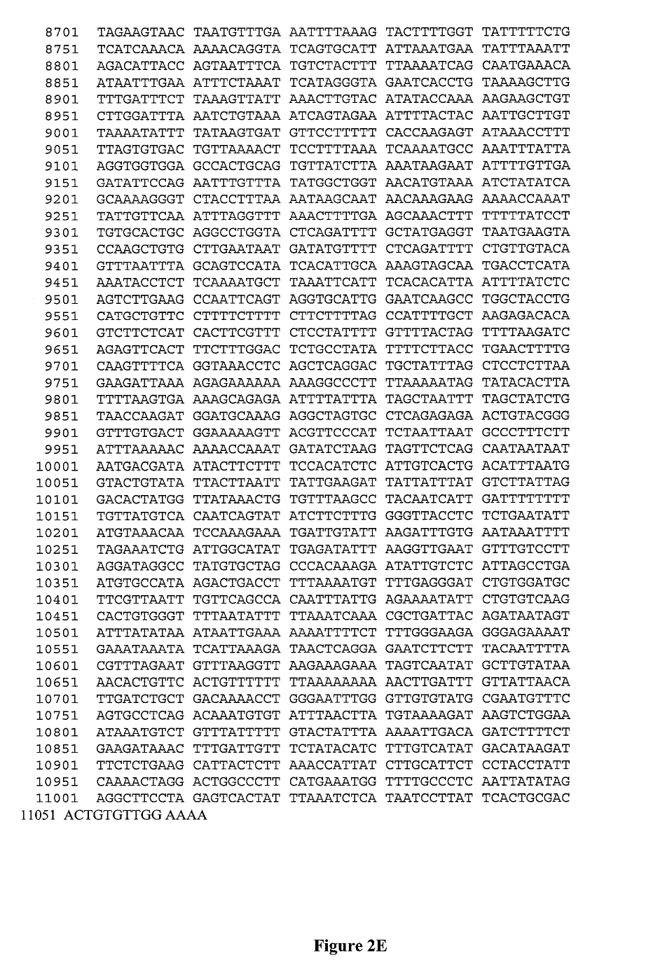

Table 3 provides a summary of the polymorphic sequences disclosed herein. The invention includes fragments of at least 15 nucleotide bases, more preferably 30 bases and even probably more 50 and the complementary sequence thereof. The nucleotide sequence can be DNA or RNA, and can be between 15 and 100 nucleotides in length. The nucleotide sequence can also be the COX-2 gene described in FIGS. 2A-2E which includes one or more polymorphisms of Table 3.

DNA fragments can be prepared, for example, by digesting plasmid DNA, or by use of PCR. Oligonucleotides for use as primers or probes are chemically synthesized by methods known in the field of the chemical synthesis of polynucleotides, including by of non-limiting example the phosphoramidite method described by Beaucage and Carruthers, Tetrahedron Lett 22.1859-1 862 (1981) and the triester method provided by Matteucci, et al J Am. Chem. Soc. 103:3185 (1981) both incorporated herein by reference. These syntheses may employ an automated synthesizer, as described in Needham-VanDevanter, D. R., et al., Nucleic Acids Res. 12:61596168(1984). Purification of oligonucleotides may be carried out by either native acrylamide gel electrophoresis or by anion-exchange HPLC as described in Pearson, J. D. and Regnier, F E., J. Chrom, 255:137-149(1983). A double stranded fragment may then be obtained, if desired, by annealing appropriate complementary single strands together under suitable conditions or by synthesizing the complementary strand using a DNA polymerase with an appropriate primer sequence. Where a specific sequence for a nucleic acid probe is given, it is understood that the complementary strand is also identified and included. The complementary strand will work equally well in situations where the target is a double-stranded nucleic acid.

The sequence of the synthetic oligonucleotide or of any nucleic acid fragment can be can be obtained using either the dideoxy chain termination method or the Maxam-Gilbert method (see Sambrook et al. Molecular Cloning—a Laboratorv Manual (2nd Ed.), Vols. 1-3, Cold Spring Harbor Laboratory, Cold Spring Harbor, N.Y., (1989), which is incorporated herein by reference. This manual is hereinafter referred to as “Sambrook et al.”; Zyskind et al., (1988)). Recombinant DNA Laboratory Manual, (Acad. Press, New York). Oligonucleotides useful in diagnostic assays are typically at least 8 consecutive nucleotides in length, and may range upwards of 18 nucleotides in length to greater than 100 or more consecutive nucleotides.

Another aspect of the invention pertains to isolated antisense nucleic acid molecules that are hybridizable to or complementary to the nucleic acid molecule comprising the SNP-containing nucleotide sequences of the invention, or fragments, analogs or derivatives thereof. An “antisense” nucleic acid comprises a nucleotide sequence that is complementary to a “sense” nucleic acid encoding a protein, e.g., complementary to the coding strand of a double-stranded cDNA molecule or complementary to an mRNA sequence. In specific aspects, antisense nucleic acid molecules are provided that comprise a sequence complementary to at least about 10, about 25, about 50, or about 60 nucleotides or a strand containing the SNP, or to only a portion thereof.

In one embodiment, an antisense nucleic acid molecule is antisense to a “coding region” of the coding strand of a polymorphic nucleotide sequence of the invention. The term “coding region” refers to the region of the nucleotide sequence comprising codons which are translated into amino acid. In another embodiment, the antisense nucleic acid molecule is antisense to a “noncoding region” of the coding strand of a nucleotide sequence of the invention. The term “noncoding region” refers to 5′ and 3′ sequences which flank the coding region that are not translated into amino acids (i.e., also referred to as 5′ and 3′ untranslated regions). The noncoding region also refers to region containing introns.

Given the coding strand sequences disclosed herein, antisense nucleic acids of the invention can be designed according to the rules of Watson and Crick or Hoogsteen base pairing. For example, the antisense nucleic acid molecule can generally be complementary to the entire coding region of an mRNA, but more preferably as embodied herein, it is an oligonucleotide that is antisense to only a portion of the coding or noncoding region of the mRNA. An antisense oligonucleotide can range in length between about 5 and about 60 nucleotides, preferably between about 10 and about 45 nucleotides, more preferably between about 15 and 40 nucleotides, and still more preferably between about 15 and 30 in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Examples of modified nucleotides that can be used to generate the antisense nucleic acid include: 5-fluorouracil, 5-bromouraci I, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, I-methylguanine, I-methylinosine, 2,2-dimethylguanine, 2-methyladenine, 2-methylguanine, 3-metbylcytosine, 5-methylcytosine, N6-adenine, 7-methylguanine, 5-methylaminomethyluracil, 5-methoxyaminomethy-2-thiouracil, beta-D-mannosylqueosine, 5′-methoxycarboxymethyluracil, 5-methoxyuracil, 2-methyltbio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subdoned in an antisense orientation (i.e., RNA transcribed from the inserted nucleic acid will be of an anti sense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a polymorphic protein to thereby inhibit expression of the protein, e.g., by inhibiting transcription and/or translation. The hybridization can be by conventional nucleotide complementary to form a stable duplex, or, for example, in the case of an anti sense nucleic acid molecule that binds to DNA duplexes, through specific interactions in the major groove of the double helix. An example of a route of administration of anti sense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of anti sense molecules, vector constructs in which the antisense nucleic acid molecule is placed under the control of a strong p0111 or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual-units, the strands run parallel to each other (Gaultier et al. (1987) Nucleic Acids Res 15: 6625-6641). T antisense nucleic acid molecule can also comprise a 2′-o-methylribonucleotide (lnoue et al. (1987) NucleicAcids Res 15:6131-6148) or a chimeric RNA-DNA analogue (Inoue et al. (1987) FEBS Lett 215: 327-330).

The invention further provides nucleotide primers which can detect polymorphisms of the invention According to another aspect of the present invention there is provided an allele specific primer capable of detecting a COX-2 polymorphism at one or more of positions 188, 502, 716, 953, 1048, 1092, 1478, 1636, 2080, 2124, 2181, 2252, 2379, 2435, 3444, 3602, 3726, 4190, 5872, 6055, 6721, 7167, 7474, 7491, or 8031 in the COX-2 gene as defined by the positions in Table 3 and FIGS. 2A-2E.

An allele specific primer is used, generally together with a constant primer, in an amplification reaction such as a PCR reaction, which provides the discrimination between alleles through selective amplification of one allele at a particular sequence position e.g. as used for ARMS™ assays. The allele specific primer is preferably 10-50 nucleotides, more preferably about 10-35 nucleotides, more preferably about 10-25 nucleotides.

An allele specific primer preferably corresponds exactly with the allele to be detected but derivatives thereof are also contemplated wherein about 6-8 of the nucleotides at the 3′, terminus correspond with the allele to be detected and wherein up to 10, such as up to 8, 6, 4, 2 or 1 of the remaining nucleotides may be varied without significantly affecting the properties of the primer.

Primers may be manufactured using any convenient method of synthesis. Examples of such methods may be found in standard textbooks, for example “Protocols for Oligonucleotides and Analogues; Synthesis and Properties,” Methods in Molecular Biology Series; Volume 20; Ed. Sudhir Agrawal, Humana ISBN: 0-89603-247-7; 1993; 1st Edition. If required the primer(s) may be labelled to facilitate detection.

According to another aspect of the present invention there is provided an allele-specific oligonucleotide probe capable of detecting a COX-2 polymorphism at one or more of positions 188, 502, 716, 953, 1048, 1092, 1478, 1636, 2080, 2124, 2181, 2252, 2379, 2435, 3444, 3602, 3726, 4190, 5872, 6055, 6721, 7167, 7474, 7491, or 8031 in the COX-2 gene as defined by the positions in Table 3 and FIGS. 2A-2E.

The allele-specific oligonucleotide probe is preferably 10-50 nucleotides, more preferably about 10-35 nucleotides, more preferably about 10-25 nucleotides.

The design of such probes will be apparent to the molecular biologist of ordinary skill. Such probes are of any convenient length such as up to 50 bases, up to 40 bases, more conveniently up to 30 bases in length, such as for example, 8-25 or 8-15 bases in length. In general such probes will comprise base sequences entirely complementary to the corresponding wild type or variant locus in the gene. However, if required one or more mismatches may be introduced, provided that the discriminatory power of the oligonucleotide probe is not unduly affected. The probes of the invention may carry one or more labels to facilitate detection.

According to another aspect of the present invention there is provided a diagnostic kit comprising an allele specific oligonucleotide probe of the invention and/or an allele-specific primer of the invention.

The diagnostic kits may comprise appropriate packaging and instructions for use in the methods of the invention. Such kits may further comprise appropriate buffer(s), nucleotides, and polymerase(s) such as thermostable polymerases, for example taq polymerase. Another method for diagnosing and identifying individuals with a polymorphism described in Table 3 is the use of an exonuclease proofreading assay taught in U.S. Pat. No. 5,391,480. Briefly, to detect a single nucleotide polymorphism in an individual or biological sample, a primer is designed and aligned with the nucleotide base of interest and a 3′ end is labeled with a fluorescent tag. If the 3′ nucleotide is a mismatch with the sample, the label is removed by an exonuclease enzyme and the label is not incorporated in the PCR product. If the primer matches the base of interest, the label base is incorporated into the PCR product. The product can then be detected or visualized using fluorescent imaging. One means of fluorescent detection is the use of fluorescent polarization machine (CRi. Inc., Woburn, Mass.).

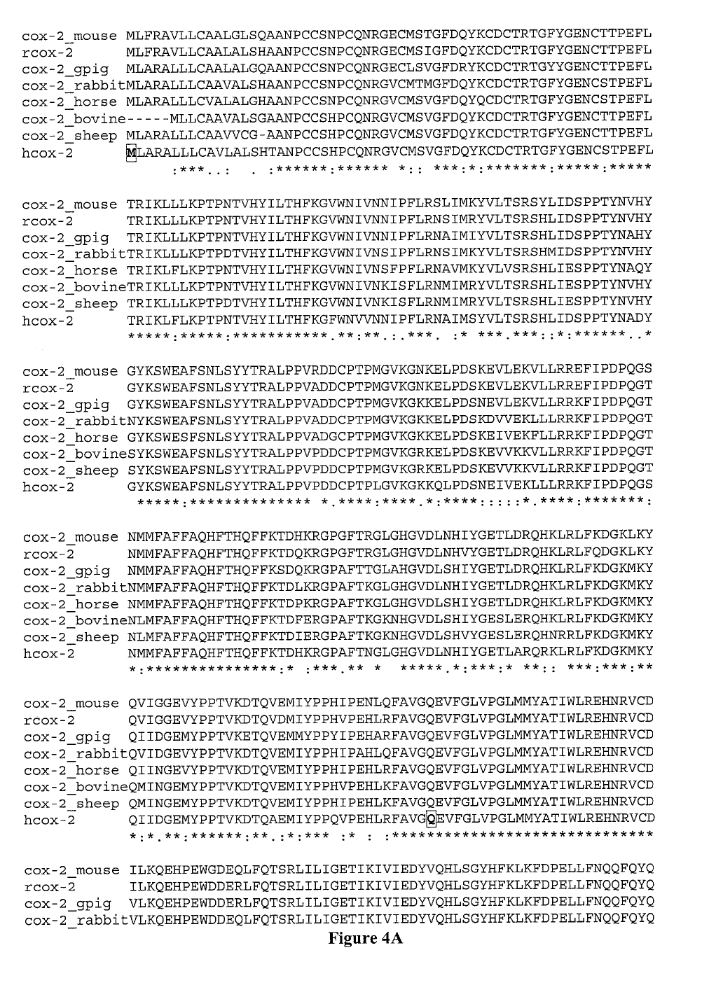

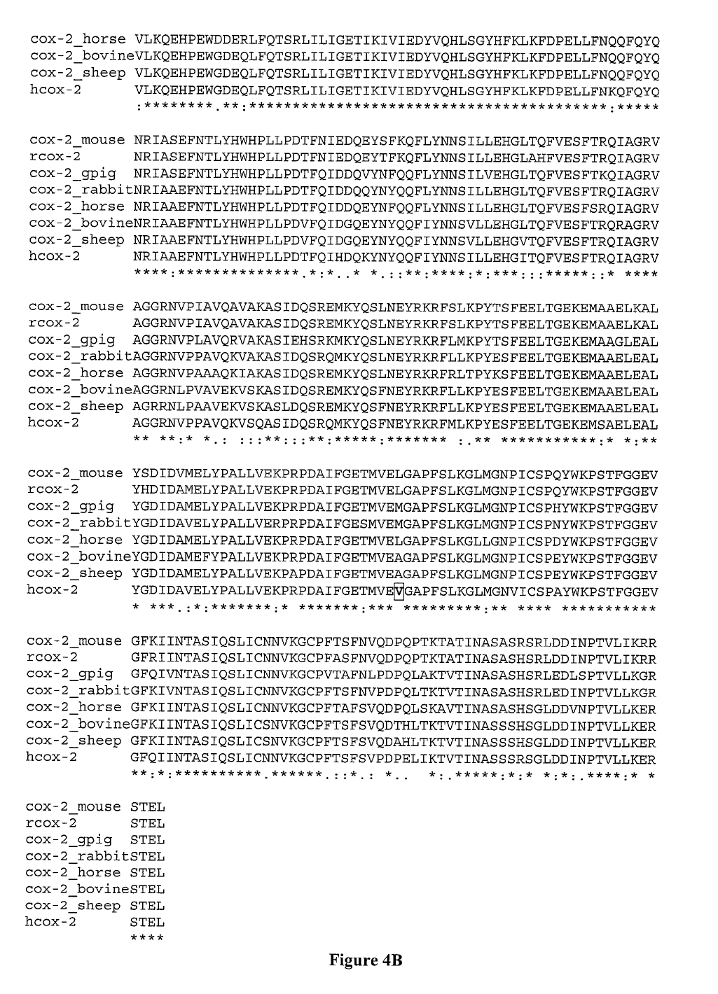

The invention also relates to polypeptide sequences of Table 3. The polypeptide can contain 5 amino acid bases, more preferably 10 bases. The polypeptide can also contain all or most of the COX-2 protein described in FIGS. 4A-B. Once DNA encoding a sequence comprising a SNP is isolated and cloned, one can express the encoded polymorphic proteins in a variety of recombinantly engineered cells. It is expected that those of skill in the art are knowledgeable in the numerous expression systems available for expression of DNA encoding a sequence of interest. No attempt to describe in detail the various methods known for the expression of proteins in prokaryotes or eukaryotes is made here.

In brief summary, the expression of natural or synthetic nucleic acids encoding a sequence of interest will typically be achieved by operably linking the DNA or cDNA to a promoter (which is either constitutive or inducible), followed by incorporation into an expression vector. The vectors can be suitable for replication and integration in either prokaryotes or eukaryotes. Typical expression vectors contain, initiation sequences, transcription and translation terminators, and promoters useful for regulation of the expression of a polynucleotide sequence of interest. To obtain high level expression of a cloned gene, it is desirable to construct expression plasmids which contain, at the minimum, a strong promoter to direct transcription, a ribosome binding site for translational initiation, and a transcription/translation terminator. The expression vectors may also comprise generic expression cassettes containing at least one independent terminator sequence, sequences permitting replication of the plasmid in both eukaryotes and prokaryotes. i.e., shuttle vectors, and selection markers for both prokaryotic and eukaryotic syszems. See Sambrook et al.

A variety of prokaryotic expression systems may be used to express the polymorphic proteins of the invention. Examples include E. coli, Bacillus, Streptomyces, and the like.

It is preferred to construct expression plasmids which contain, at the minimum, a strong promoter to direct transcription, a ribosome binding site for translational initiation, and a transcription/translation terminator. Examples of regulatory regions suitable for this purpose in E. coli are the promoter and operator region of the E. coli tryptophan biosynthetic pathway as described by Yanofsky, C., J. Bacterial. 158:1018-1024(1984) and the leftward promoter of phage lambda (P ) as described by A, I. and Hagen, D. Ann. Rev. Genet. 14:399-445 (1980). The inclusion of selection markers in DNA vectors transformed in E. coli is also useful. Examples of such markers include genes specifying resistance to ampicillin, tetracycline, or chloramphenicol. See Sambrook et al. for details concerning selection markers for use in E. coli.

To enhance proper folding of the expressed recombinant protein, during purification from E. coli, the expressed protein may first be denatured and then renatured. This can be accomplished by solubilizing the bacterially produced proteins in a chaotropic agent such as guanidine HCI and reducing all the cysteine residues with a reducing agent such as beta-mercaptoethanol. The protein is then renatured, either by slow dialysis or by gel filtration. See U.S. Pat. No. 4,511,503. Detection of the expressed antigen is achieved by methods known in the art as radioimmunoassay, or Western blotting techniques or immunoprecipitation. Purification from E. coli can be achieved following procedures such as those described in U.S. Pat. No. 4,511,503.

Any of a variety of eukaryotic expression systems such as yeast, insect cell lines, bird, fish, and mammalian cells, may also be used to express a polymorphic protein of the invention. As explained briefly below, a nucleotide sequence harboring a SNP may be expressed in these eukaryotic systems. Synthesis of heterologous proteins in yeast is well known. Methods in Yeast Genetics, Sherman, F., et al., Cold Spring Harbor Laboratory, (1982) is a well recognized work describing the various methods available to produce the protein in yeast. Suitable vectors usually have expression control sequences, such as promoters, including 3-phosphogtycerate kinase or other glycolytic enzymes, and an origin of replication, termination sequences and the like as desired. For instance, suitable vectors are described in the literature (Botstein, et al.,Gene 8:17-24 (1979); Broach, et al., Gene 8:121-133 (1979)).

Two procedures are used in transforming yeast cells. In one case, yeast cells are first converted into protoplasts using zymolyase, lyticase or glusulase, followed by addition of DNA and polyethylene glycol (PEG). The PEG-treated proloplasts are then regenerated in a 3% agar medium under selective conditions. Details of this procedure are given in the papers by J. D. Beggs, Nature (London) 275:104-109 (1978); and Hinnen, A., et al., Proc. Nati. Acad. Sci. USA, 75:1929-1933 (1978). The second procedure does not involve removal of the cell wall. Instead the cells are treated with lithium chloride or acetate and PEG and put on selective plates (Ito, H., et al., J. Bact, 153163-168 (1983)). Cells and applying standard protein isolation techniques to the lysates.

The purification process can be monitored by using Western blot techniques or radio immunoassay or other standard techniques. The sequences encoding the proteins of the invention can also be ligated to various immunoassay expression vectors for use in transforming cell cultures of; for instance, mammalian, insect, bird or fish origin. Illustrative of cell cultures useful for the production of the polypeptides are mammalian cells. Mammalian cell systems often will be in the form of monolayers of cells although mammalian cell suspensions may also be used. A number of suitable host cell lines capable of expressing intact proteins have been developed in the art, and include the HEK293, BHK21, and CHO cell lines, and various human cells such as COS cell lines, HeLa cells, myeloma cell lines, Jurkat cells, etc. Expression vectors for these cells can include expression control sequences, such as an origin of replication, a promoter (e.g., the CMV promoter, a HSV tk promoter or pgk (phosphoglycerate kinase) promoter), an enhancer (Queen et al. Immunol. Rev. 89:49 (1986)) and necessary processing information sites, such as ribosome binding sites, RNA splice sites, polyadenylation sites (e.g., an SV4O large T Ag poly A addition site), and transcriptional terminator sequences.

Other animal cells are available, for instance, from the American Type Culture Collection Catalogue of Cell Lines and Hybridomas (7th edition, (1992)). Appropriate vectors for expressing the proteins of the invention in insect cells are usually derived from baculovirus. Insect cell lines include mosquito larvae, silkworm, armyworm, moth and Drosophila cell lines such as a Schneider cell line (See Schneider J. Embryol. Exp. Morphol., 27:353-365 (1987). As indicated above, the vector, e.g., a plasmid, which is used to transform the host cell, preferably contains DNA sequences to initiate transcription and sequences to control the translation of the protein. These sequences are referred to as expression control sequences. As with yeast, when higher animal host cells are employed, polyadenylation or transcription terminator sequences from known mammalian genes need to be incorporated into the vector. An example of a terminator sequence is the polyadenylation sequence from the bovine growth hormone gene. Sequences for accurate splicing of the transcript may also be included. An example of a splicing sequence is the VPI intron from SV4O (Sprague, J. et a!., J. Virol. 45: 773-781 (1983)). Additionally, gene sequences to control replication in the host cell may be Saveria Campo, M., 1985, “Bovine Papilloma virus DNA a Eukaryotic Cloning Vector” in DNA Cloning Vol.11 a Practical AnDroach Ed. D. M. Glover, IRL Press, Arlington, Va. pp. 213-238. The host cells are competent or rendered competent for transformation by various means. There are several well-known methods of introducing DNA into animal cells. These include: calcium phosphate precipitation, fusion of the recipient cells with bacterial protoplasts containing the DNA, treatment of the recipient cells with liposomes containing the DNA, DEAE dextran, electroporation and micro-injection of the DNA directly into the cells.

The transformed cells are cultured by means well known in the art (Biochemical Methods in Cell Culture and Virology, Kuchler, R. J., Dowden, Hutchinson and Ross, Inc., (1977)). The expressed polypeptides are isolated from cells grown as suspensions or as monolayers. The latter are recovered by well known mechanical, chemical or enzymatic means.

General methods of expressing recombinant proteins are also known and are exemplified in R. Kaufman, Methods in Enzymology 185, 537-566 (1990). As defined herein “operably linked” refers to linkage of a promoter upstream from a DNA sequence such that the promoter mediates transcription of the DNA sequence. Specifically, “operably linked” means that the isolated polynucleotide of the invention and an expression control sequence are situated within a vector or cell in such a way that the gene encoding the protein is expressed by a host cell which has been transformed (transfected) with the ligated polynucleotide/expression sequence. The term “vector”, refers to viral expression systems, autonomous self-replicating circular DNA (plasmids), and includes both expression and nonexpression plasmids.

A number of types of cells may act as suitable host cells for expression of the protein. Mammalian host cells include, for example, monkey COS cells, Chinese Hamster Ovary (CHO) cells, Human kidney 293 cells, human epdiermal A431 cells, human Co10205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells. Alternatively, it may be possible to produce the protein in lower eukaryotes such as yeast or in prokaryotes such as bacteria. Potentially suitable yeast strains include Saccharomyces cerevisiae, Schizosaccharomyces pombe, Kluyveromyces strains, Candida or any yeast strain capable of expressing heterologous proteins. Potentially suitable bacteral strains include Escherichia coli, Bacillus sublilis, Salmonella typhimuri urn, or any bacterial strain capable of expressing heterologous proteins. If the protein is made in yeast or bacteria, it may be necessary to modify the protein produced therein, for example by phosphorylation or glycosyjation of the appropriate sites, in order to obtain the functional protein.

The protein may also be produced by operably linking the isolated polynucleotide of the invention to suitable control sequences in one or more insect expression vectors, and employing an insect expression system. Materials and methods for baculovirus/insect cell expression systems are commercially available in kit form from, e.g., Invitrogen, San Diego, Calif., U.S.A. (the MaxBacOc kit), and such methods are well known in the art, as described in Summers and Smith, Texas Agricultural Experiment Station Bulletin No. 1555 (1987), incorporated herein by reference. As used herein, an insect cell capable of expressing a polynucleotide of the present invention is “transformed.” The protein of the invention may be prepared by culturing transformed host cells under culture conditions suitable to express the recombinant protein.

The polymorphic protein of the invention may also be expressed as a product of transgenic animals, e.g., as a component of the milk of transgenic cows, goats, pigs, or sheep which are characterized by somatic or germ cells containing a nucleotide sequence encoding the protein. The protein may also be produced by known conventional chemical synthesis. Methods for constructing the proteins of the present invention by synthetic means are known to those skilled in the art.

The polymorphic proteins produced by recombinant DNA technology may be purified by techniques commonly employed to isolate or purify recombinant proteins. Recombinantly produced proteins can be directly expressed or expressed as a fusion protein. The protein is then purified by a combination of cell lysis (e.g. sonication) and affinity chromatography. For fusion products, subsequent digestion of the fusion protein with an appropriate proteolytic enzyme releases the desired polypeptide. The polypeptides of this invention may be purified to substantial purity by standard techniques well known in the art, including selective precipitation with such substances as ammonium sulfate, column chromatography, immunopurification methods, and others. See, for instance, R. Scopes, Protein Purification: Principles and Practice, Springer-Verlag: New York (1982), incorporated herein by reference. For example, in an embodiment, antibodies may be raised to the proteins of the invention as described herein. Cell membranes are isolated from a cell line expressing the recombinant protein, the protein is extracted from the membranes and immunoprecipitated. The proteins may then be further purified by standard protein chemistry techniques as described above.

The resulting expressed protein may then be purified from such culture (i.e., from culture medium or cell extracts) using known purification processes, such as gel filtration and ion exchange chromatography. The purification of the protein may also include an affinity column containing agents which will bind to the protein; one or more column steps over such affinity resins as concanavalin A-agarose, heparin-Toyopearl or Cibacrom blue 3GA Sepharose B; one or more steps involving hydrophobic interaction chromatography using such resins as phenyl ether, butyl ether, or propyl ether; or immuno affinity chromatography. Alternatively, the protein of the invention may also be expressed in a form which will facilitate purification. For example, it may be expressed as a fusion protein, such as those of maltose binding protein (MBP), glutathione-S-transferase (GST) or thioredoxin (TRX). Kits for expression and purification of such fusion proteins are commercially available from New England BioLab (Beverly, Mass.), Pharmacia (Piscataway, N.J.) and InVitrogen, respectively. The protein can also be tagged with an epitope and subsequently purified by using a specific antibody directed to such epitope. One such epitope (“Flag”) is commercially available from Kodak New Haven, Conn.). Finally, one or more reverse-phase high performance liquid chromatography (RI)- HPLC) steps employing hydrophobic RP-HPLC media, e.g., silica gel having pendant methyl or other aliphatic groups, can be employed to further purify the protein. Some or all of the foregoing purification steps, in various combinations, can also be employed to provide a substantially homogeneous isolated recombinant protein. The protein thus purified is substantially free of other mammalian proteins and is defined in accordance with the present invention as an “isolated protein.”

The term “antibody” as used herein refers to immunoglobulin molecules and immunologically active portions of immunoglobulin molecules, i.e., molecules that contain an antigen binding site that specifically binds (immunoreacts with) an antigen, such as polymorphic. Such antibodies include, but are not limited to, polyclonal, monoclonal, chimeric, single chain, Fab and F(ab′)2 fragments, and an Fab expression library. In a specific embodiment, antibodies to human polymorphic proteins are disclosed.

The phrase “specifically binds to”, “immunospecifically binds to” or is “specifically immunoreactive with”, an antibody when referring to a protein or peptide, refers to a binding reaction which is determinative of the presence of the protein in the presence of a heterogeneous population of proteins and other biological materials. Thus, for example, under designated immunoassay conditions, the specified antibodies bind to a particular protein and do not bind in a significant amount to other proteins present in the sample. Specific binding to an antibody under such conditions may require an antibody that is selected for its specificity for a particular protein. Of particular interest in the present invention is an antibody that binds immunospecifically to a polymorphic protein but not to its cognate wild type allelic protein, or vice versa. A variety of immunoassay formats may be used to select antibodies specifically immunoreactive with a particular protein. For example, solid-phase ELI SA immunoassays are routinely used to select monoclonal antibodies specifically immunoreactive with a protein. See Harlow and Lane (1988) Antibodies, a Laboratory Manual, Cold Spring Harbor Publications, New York, for a description of immunoassay formats and conditions that can be used to determine specific immunoreactivity.

Polyclonal and/or monoclonal antibodies that immunospecifically bind to polymorphic gene products but not to the corresponding prototypical or “wild-type” gene products are also provided. Antibodies can be made by injecting mice or other animals with the variant gene product or synthetic peptide. Monoclonal antibodies are screened as are described, for example, in Harlow & Lane, Antibodies, A Laboratory Manual, Cold Spring Harbor Press, New York (1988); Goding, Monoclonal antibodies, Principles and Practice (2d ed.) Academic Press, New York (1986). Monoclonal antibodies are tested for specific immunoreactivity with a variant gene product and lack of immunoreactivity to the corresponding prototypical gene product.

An isolated polymorphic protein, or a portion or fragment thereof; can be used as an immunogen to generate the antibody that bind the polymorphic protein using standard techniques for polyclonal and monoclonal antibody preparation. The full-length polymorphic protein can be used or, alternatively, the invention provides antigenic peptide fragments of polymorphic for use as immunogens. The antigenic peptide of a polymorphic protein of the invention comprises at least 5 amino acid residues of the amino acid sequence encompassing the polymorphic amino acid and encompasses an epitope of the polymorphic protein such that an antibody raised against the peptide forms a specific immune complex with the polymorphic protein. Preferably, the antigenic peptide comprises at least 10 amino acid residues, more preferably at least 15 amino acid residues, even more preferably at least 20 amino acid residues, and most preferably at least 30 amino acid residues. Preferred epitopes encompassed by the antigenic peptide are regions of polymorphic that are located on the surface of the protein, e.g., hydrophilic regions.

For the production of polyclonal antibodies, various suitable host animals (e.g., rabbit, goat, mouse or other mammal) may be immunized by injection with the polymorphic protein. An appropriate immunogenic preparation can contain, for example, recombinantly expressed polymorphic protein or a chemically synthesized polymorphic polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Cozynebacterium parvum, or similar immunostimulatory agents. If desired, the antibody molecules directed against polymorphic proteins can be isolated from the mammal (e.g., from the blood) and further purified by well known techniques, such as protein A chromatography, to obtain the IgG fraction.

The term “monoclonal antibody” or “monoclonal antibody composition”, as used herein, refers to a population of antibody molecules that originates from the clone of a singly hybridoma cell, and that contains only one type of antigen binding site capable of immunoreacting with a particular epitope of a polymorphic protein. A monoclonal antibody composition thus typically displays a single binding affinity for a particular polymorphic protein with which it immunoreacts. For preparation of monoclonal antibodies directed towards a particular polymorphic protein, or derivatives, fragments, analogs or homologs thereof; any technique that provides for the production of antibody molecules by continuous cell line culture may be utilized. Such techiuques include, but are not limited to, the hybridoma technique (see Kohler & Milstein, 1975 Nature 256: 495-497); the trioma technique; the human B-cell hybridoma technique (see Kozbor, et al., 1983 Immunol Today 4: 72) and the EBV hybridoma technique to produce human monoclonal antibodies (see Cole, et aL, 1985 In: MONOCLONAL ANTIBODIES AND CANCER THERAPY, Alan R. Liss, Inc., pp.77-96). Human monoclonal antibodies may be utilized in the practice of the present invention and may be produced by using human bybridomas (see Cote et al., 1983. Proc NatlAcadSci USA go: 2026-2030) or by transforming human B-cells with Epstein Barr Virus in vitro (sec Cole, ef aL, I 985 In: MONOCLONAL ANTIBODIES AND CANCER THERAPY, Alan R. Liss, Inc., pp.77-96).

According to the invention, techniques can be adapted for the production of single-chain antibodies specific to a polymorphic protein (see e.g., U.S. Pat. No. 4,946,778), in addition, methodologies can be adapted for the construction of Fab expression libraries (see e.g., Huse, et al., 1989 Science 246:1275-1281) to allow rapid and effective identification of monoclonal Fab fragments with the desired specificity for a polymorphic protein or derivatives, fragments, analogs or homologs thereof. Non-human antibodies can be “humanized” by techniques well known in the art. See e.g., U.S. Pat. No.5,225,539. Antibody fragments that contain the idiotypes to a polymorphic protein may be produced by techniques known in the art including, but not limited to: (i) an F(ab′)2 fragment produced by pepsin digestion of an antibody molecule; (ii) an Fab fragment generated by reducing the disuWide bridges of an F(ab)₂ fragment; (iii) an Fab fragment generated by the treatment of the antibody molecule with papain and a reducing agent and (iv) Fv fragments.

Additionally, recombinant anti-polymorphic protein antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT International Application No. PCT/U586102269; European Patent Application No. 184,187; European Patent Application No. 171,496; European Patent Application No. 173,494; PCT International Publication No. WO 86/01533; U.S. Pat. No. 4,816,567; European Patent Application No. 125,023; Better et al. (1988) Science 240:1041-1043; Liu et al. (1987) PNAS 84:3439-3443; Liu et al. (1987)] immunol. 139:3521-3526; Sun et al. (1987) PNAS 84:214-218; Nishimura et al. (1987) Cancer Res 47:999-1005; Wood et al. (1985) Nature 314:446-449; Shaw et al. (1988)] Natl. Cancer Inst 80:1553-1559); Morrison(I 985) Science 229:1202-1207; Oi et al. (1986) BioTechniques 4:214; U.S. Pat. No.5,225,539; Jones e tal. (1986) Nature 321:552-525; Verhoeyan et al. (1988) Science 239:1534; and Beidler et al. (1988) Jlmmunol 141:4053-4060.

In one embodiment, methodologies for the screening of antibodies that possess the desired specificity include, but are not limited to, enzyme-linked immunosorbent assay (ELISA) and other immunologically-mediated techniques known within the art. In another aspect of the invention, the single nucleotide polymorphisms of this invention may be used as genetic markers in linkage studies.

According to another aspect of the present invention there is provided a method of treating a human in need of treatment for a disorder or disease involving COX-2 protein activity with a COX-2 drug. Such disorders include fever, arthritis including rheumatoid arthritis, Alzheimer's disease, seizures, bone conditions including osteoarthritis and other various inflammatory conditions. COX has also been shown to be involved in cancer and in particular, breast cancer. The method comprises: i) identification of a single nucleotide polymorphism in the COX-2 gene in the human, which identification comprises determining the sequence of the nucleic acid at one or more of positions 188, 502, 716, 953, 1048, 1092, 1478, 1636, 2080, 2124, 2181, 2252, 2379, 2435, 3444, 3602, 3726, 4190, 5872, 6055, 6721, 7167, 7474, 7491, or 8031 in the COX-2 gene as defined by the positions in Table 3 and FIGS. 2A-2E, and ii) determining the status of the human by reference to polymorphisms in the COX-2 gene; and iii) administering an effective amount of a COX-2 drug, the amount and for type of drug being determined by (ii) above.

Preferably determination of the status of the human is clinically useful especially in the field of pharmacogenomics. Examples of clinical usefulness include deciding which drug or drugs to administer and/or establishing the effective amount of the drug or drugs. Another embodiment of the invention is predicting a clinical response to a therapeutic compound. Based on the polymorphisms of an individual, the response to a particular COX-2 drug can be predicted. The type of practice is sometimes referred to as patient profiling. Based on the patient's genotype or haplotype the clinical response to a particular compound can be predicted.

Many COX-2 inhibitors have been disclosed in the following publication: Hawkey, LANCET, 353: 307-314 (1999). COX-2 inhibitors are of value in a number of disease conditions or sicknesses, including fever, arthritis including rheumatoid arthritis, Alzheimer's disease, seizures, bone conditions including osteoarthritis and other various inflammatory conditions. In addition, COX-2 drugs have been shown to prevent breast cancer (Harris et al., Cancer Research 60:2101-03 (2000)). Therefore, identification of an individual with a particular COX-2 polymorphism could help determine which drug and the proper dose of that drug based on the polymorphism of COX-2 in order to increase the efficacy of the drug as well as reduce any possible side effects.

According to another aspect of the present invention there is provided use of a COX-2 drug in the preparation of a medicament for treating a COX-2 mediated disease in a human diagnosed as having a single nucleotide polymorphism at one or more of positions 188, 502, 716, 953, 1048, 1092, 1478, 1636, 2080, 2124, 2181, 2252, 2379, 2435, 3444, 3602, 3726, 4190, 5872, 6055, 6721, 7167, 7474, 7491, or 8031 in the COX-2 gene as defined by the positions in Table 3 and FIGS. 2A-2E.

According to another aspect of the present invention there is provided a pharmaceutical pack comprising COX-2 drug and instructions for administration of the drug to humans diagnostically tested for a single nucleotide polymorphism at one or more of positions 188, 502, 716, 953, 1048, 1092, 1478, 1636, 2080, 2124, 2181, 2252, 2379, 2435, 3444, 3602, 3726, 4190, 5872, 6055, 6721, 7167, 7474, 7491, or 8031 in the COX-2 gene as defined by the positions in Table 3 and FIGS. 2A-2E. For additional information relating to the individual SNPs, see Example 1.

In yet another embodiment, the invention provides polymorphisms, which may indicate if an individual is predisposed to certain disease mediated by COX-2 activity. By identifying such individuals using the methods described herein, a preventive regimen can be prescribed to prevent such disorders or diseases from manifesting.

The invention also relates to identifying individuals using the nucleic acid sequences provided herein. The compilation of polymorphic sites in an individual distinguishes that individual from others in a population. See generally National Research Council, The Evaluation of Forensic DNA Evidence (Eds. Pollard et al., National Academy Press, DC, 1996). These polymorphisms provide a unique set of markers which can be useful for forensic analysis. For example, one can determine whether a blood sample collected from a crime scene matches blood sample from a suspect by determining if one or more polymorphisms are the same in both samples. One can perform statistical analysis to determine the probability that a match of suspect and crime scene sample would occur by chance. For further teaching see U.S. Pat. No. 5,856,1904 and WO 95/12607.

Similar to the forensic analysis above, paternity testing in determining whether a male is the father of a child could also be accomplished by the use of the nucleic acid sequence provided herein. Polymorphic sites as described above can be used in distinguishing individuals. The probability of parentage exclusion represents the probability that a random male will have a polymorphic form at a given polymorphic site makes him incompatible as the father. These statistical analyses are taught in WO 95/12607.

According to another aspect of the present invention there is provided a computer readable medium comprising at least one polynucleotide sequence of the invention stored on the medium. The computer readable medium may be used, for example, in homology searching, mapping, haplotyping, genotyping or pharmacogenetic analysis or any other bioinformatic analysis. The reader is referred to Biomformatics, A practical guide to the analysis of genes and proteins, Edited by A D Baxevanis & B F F Quellette, John Wiley & Sons, 1988. Any computer readable medium may be used, for example, compact disk, tape, floppy disk, hard drive or computer chips.

The polynucleotide sequences of the invention, or parts thereof, particularly those relating to and identifying the single nucleotide polymorphisms identified herein represent avaluable information source, for example, to characterize individuals in terms of haplotype and other sub-groupings, such as investigation of susceptibility to treatment with particular drugs. These approaches are most easily facilitated by storing the sequence information in a computer readable medium and then using the information in standard bioinformatics programs or to search sequence databases using state of the art searching tools such as “GCG”. Thus, the polynucleotide sequences of the invention are particularly useful as components in databases useful for sequence identity and other search analyses. As used herein, storage of the sequence information in a computer readable medium and use in sequence databases in relation to ‘polynucleotide or polynucleotide sequence of the invention’ covers any detectable chemical or physical characteristic of a polynucleotide of the invention that may be reduced to, converted into or stored in a tangible medium, such as a computer disk, preferably in a computer readable form. For example, chromatographic scan data or peak data, photographic scan or peak data, mass spectrographic data, sequence gel (or other) data.

The invention provides a computer readable medium having stored thereon one or more polynucleotide sequences of the invention. For example, a computer readable medium is provided comprising and having stored thereon a member selected from the group consisting of: a polynucleotide comprising the sequence of a polynucleotide of the invention, a polynucleotide consisting of a polynucleotide of the invention, a polynucleotide which comprises part of a polynucleotide of the invention, which part includes at least one of the polymorphisms of the invention, a set of polynucleotide sequences wherein the set includes at least one polynucleotide sequence of the invention, a data set comprising or consisting of a polynucleotide sequence of the invention or a part thereof comprising at least one of the polymorphisms identified herein.

A computer based method is also provided for performing sequence identification, said method comprising the steps of providing a polynucleotide sequence comprising a polymorphism of the invention in a computer readable medium; and comparing said polymorphism containing polynucleotide sequence to at least one other polynucleotide or polypeptide sequence to identify identity (homology), i.e. screen for the presence of a polymorphism.

### EXAMPLE I

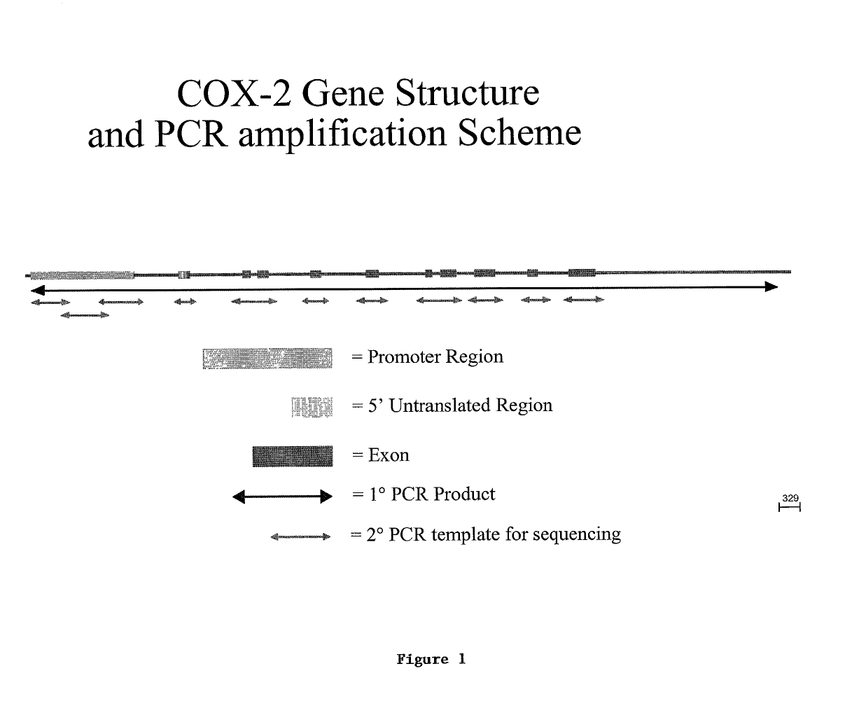

Templates for sequencing were generated by primary polymerase chain reaction (PCR) amplification of the entire gene for COX2, followed by secondary PCR amplification of twelve smaller overlapping fragments using chimeric primers. FIG. 1 illustrates this approach.

The conditions for the primary PCR reaction were as follows: 25 ng of genomic DNA, 500 μM of each primary primer, 300 nM dNTPs, 1X Boehringer-Mannheim Expand™ Long PCR Buffer 3 and 1 unit Boehringer-Mannheim Expand™ Long PCR polymerase were used in a final volume of 25 μl. Amplification was carried out under the following cycling conditions: initial denaturation of 94° C. for 1 minutes, followed by 35 cycles of 94° C. for 10 seconds, 68° C. for 14:30 minutes with an additional 5 secs/cycle. A final elongation step of 68° for 10 minutes was carried out followed by storage at 4° C. The primers for the primary PCR are listed in Table 1.

TABLE 1
Primers for primary PCR

1° Forward Primer:
5′-ATTTAATACACTCCCATGACCAGCATCCCA-3′
1° Reverse Primer:
5′-TTTTCCAACACAGTGTCGCAGTGAATAAGG-3′

The product of the primary PCR reaction was then diluted 1:100 with sterile water and 5 μl used in nested PCR reactions under the same conditions as described above, with the following exceptions: 350 nM dNTP and Buffer 1 were used in the reaction mix, and 32 cycles of 94° for 10 seconds, 55° for 30 seconds and 68° for 30 seconds. The primers for the nested or secondary PCR are listed in Table 2.

TABLE 2
M13 Chimeric PCR primers for amplification of
COX-2 overlapping fragments:
(M13F or M13R portions are underlined)

COX-2_N1F:
5′-TGTAAAACGACGGCCAGTTAATACACTCCCATGACCAG-3′
COX-2_N1R:
5′-AGGAAACAGCTATGACCATTTTCAACTAAGAGCGTGGAT-3′
COX-2_N2F:
5′-TGTAAAACGACGCCAGTTTCTAACATGGCTTCTAACC-3′
COX-2_N2R:
5′-AGGAAACAGCTATGACCATTCCAAGATTATGAGTTGTGA-3′
COX-2_N3F:
5′-TGTAAAACGACGGCCAGTAAAAGCAAAGATGAAATTCC-3′
COX-2_N3R:
5′-AGGAAACAGCTATGACCATGCGAGTAAGGTTAAGAAAGG-3′
COX-2_N4F:
5′-TGTAAAACGACGGCCAGTGAAGAAGAAAAGACATCTGG-3′
COX-2_N4R:
5′-AGGAAACAGCTATGACCATTCACGTAGCTTCTCTATTCG-3′
COX-2_N5F:
5′-TGTAAAACGACGGCCAGTGTTTTATCCATTCTAAGGCA-3′
COX-2_N5R:
5′-AGGAAACAGCTATGACCATTATTTTTGGCGATTAAGATG-3′
COX-2_N6F:
5′-TGTAAAACGACGGCCAGTTTTGGAGTTACATTCAACCT-3′
COX-2_N6R:
5′-AGGAAACAGCTATGACCATCCCATAGATAACCATGCTAA-3′
COX-2_N7F:
5′-TGTAAAACGACGGCCAGTGATTGACAGTCACCATCTCC-3′
COX-2_N7R:
5′-AGGAAACAGCTATGACCATTAGCCCTTGACTATGATTTG-3′
COX-2_N8F:
5′-TGTAAAACGACGGCCAGTATTAATTAGCAATTCATGG-3′
COX-2_N8R:
5′-AGGAAACAGCTATGACCATGTGAGTTTTCATTTACCACA-3′
COX-2_N9F:
5′-TGTAAAACGACGGCCAGTGAAGAAAACAGAAATGAAGG-3′
COX-2_N9R:
5′-AGGAAACAGCTATGACCATTTCAAACAAAGTTAGGCTTC-3′
COX-2_N10F:
5′-TGTAAAACGACGGCCAGTTGTTGAAATGTAGGTAAGCA-3′
COX-2_N10R:
5′-AGGAAACAGCTATGACCATAGCTACTCAGGAAGTTGAGG-3′
COX-2_N11F:
5′-TGTAAAACGACGGCCAGTAAATGGAAACAGAGAAGTTG-3′
COX-2_N11R:
5′-AGGAAACAGCTATGACCATCAAGTATGACTCCTTTCTCC-3′

Following secondary PCR, the products from each individual were then sequenced to identify polymorphisms of the COX-2 gene. Each PCR product was diluted 1:25 and then was sequenced using DYEnamic Energy Transfer Primer Kits (AmershamPharmacia Biotech, Piscataway, N.J.). Briefly, all reactions were performed in 96 well trays. Four separate reactions, one each for A, C, G, and T, were performed for each template. Each reaction included 2 μl of the sequencing reaction mix and 3 μl of diluted template. The plates were then heat sealed with foil tape and placed in a thermal cycler and cycled according to the manufacturer's recommendation. After cycling the four reactions (A,C,G and T) were pooled. 3 μl of the pooled product was transferred to a new 96 well plate and 1 μl of the manufacturer's loading dye was added to each well. 1 μl of pooled material was directly loaded onto a 48 lane gel running on an ABI 377 DNA sequencer (Palo Alto, Calif.) for 10 hour at 2.4 kV.

The analysis of the sequencing gel followed. The computer program, Polyphred (university of Washington, Seattle, Wash.) was used to assemble sequence sets for viewing with Consed (University of Washington, Seattle, Wash.), another computer program. All sequences for each study subject were assembled in a unique directory along with a monochromosomal sequence set and a color annotated reference sequence. Polyphred indicates potential polymorphic sites with purple and red tags. Two independent readers were used to examine each sequence set and assessed the validity of each tagged site.

The polymorphism for the individuals sequenced are listed in Table 3. A total of twenty-five polymorphisms were discovered. The first column in Table 3 list the SNP identification number. The second column contains the nucleotide base substitution from the reference sequence. The location of the polymorphims are based on Genbank submission AC number AL033533 which is listed in the third column. The respective amino change of the COX-2 protein is listed in the fourth column.

TABLE 3
SNPs of COX-2

| | Base exchange | Position in functional part (first | |
|---|---|---|---|
| | relative base identity of | base of structural gene is position | |
| SNP | reference sequence | 2377) | Amino acid exchange |
| 1 | A to G | 188, promoter region | No |
| 2 | A to G | 503, promoter region | No |
| 3 | A to C | 716, promoter region | No |
| 4 | A to G | 953, promoter region | No |
| 5 | A to G | 1048, promoter region | No |
| 6 | A to G | 1092, promoter region | No |
| 7 | G to C | 1478, promoter region | No |
| 8 | T to C | 1636, promoter region | No |
| 9 | C to G | 2080, promoter region | No |
| 10 | T to C | 2124, promoter region | No |
| 11 | C to G | 2181, promoter region | No |
| 12 | T to G | 2252, promoter region | No |
| 13 | G to A | 2379, exon 1 (ATG start codon) | Met 1-S to Ile 1-S |
| 14 | A to C | 2435, intron 1 | No |
| 15 | C to G | 3444, intron 2 | No |
| 16 | G to C | 3602, intron 2 | No |
| 17 | G to C | 3726, intron 2 | No |
| 18 | T to C | 4190, intron 2 | No |
| 19 | G to A | 5872, intron 5 | No |
| 20 | G to C | 6055, exon 6 | Gln 257 to His |
| 21 | T to C | 6721, exon 7 | no, |
| | | | codon impact His 403 |
| 22 | C to T | 7167, intron 7 | No |
| 23 | T to C | 7474, intron 8 | No |
| 24 | G to T | 7491, intron 8 | No |
| 25 | T to C | 8031, exon 9 | Val 511 to Ala |

Impact of Nucleotide Polymorphisms and Base Insertions on Function of COX-2 Gene and COX-2 Protein:

Polymorphisms labeled, SNP 1-12 of Table 3 are located in the 5′ untranslated region of the COX-2 gene at positions 188, 503, 716, 953, 1048, 1092, 1478, 1636, 2080, 2124, 2181 and 2252, respectively. All base exchanges, either per se or in combination of each other could change the basal as well as the induced expression of that gene. As shown in FIGS. 2A-2E, there are multiple sequence motifs in the 5′ untranslated region of COX-2 which are regulatory sequences or protein binding sites (Hla and Neilson, 1992).

GEMS Launcher (Genomatix, Munich/Germany) was used to search for known promoter modules (one or more promoter consensus sequences positioned within functional spacing). The following four elements were discovered:

1.) AP1F/NFAT (at position 176-190 and at position 223-241). This module was characterized upstream of the human granulocyte-macrophage colony stimulating factor gene and is required for the induction of the gene during T-cell activation (Masuda et al., 1993. Mol Cell Biol 13, 7399-7407).

2.) AP1F/ETSF (at position 694-708). Module found upstream of the human macrophage scavenger receptor gene. It synergistically increases the transcription from the respective promoter (Wu et al., Mol Cell Biol 14, 2129-2139(1994)).

3.) EGRF/NFAT (at position 893-925). Element in front of the human interleukin-2 gene. EGR and NFAT synergistically induce transcription from the interleukin-2 promoter (Decker et al., J Biol Chem 273, 26923-26930, (1998)).

4.) CEBP/NFKB (at position 1700-1804). Module controlling the human acute phase serum amyloid A protein (SAA2.2) gene. It mediates the synergistic response of the SA A2.2 gene promoter to the cytokines interleukin-1 and interleukin-6 (Betts et al., J Biol Chem 268, 25624-25631(1993)).

Any base change within or near these promoter modules can impact transcriptional control and inducibility of the COX-2 gene, especially if base exchanges will impact interaction of transcriptional factors/proteins with the nucleic acid. In particular, SNP 1 at position 188 will considerably weaken a NFAT binding site (see FIGS. 2A-2E)

SNP 13 is located at position 2379 of the coding sequence (exon 1) and affects the coding sequence directly by changing the ATG start codon to ATA. Thus, the initiator methionine (methionine 1-signal=M1-S), which is strictly required for translation initiation, is altered to isoleucine. This base alteration will not allow translation, therefore resulting in a defective gene. A second methionine codon (ATG) is coding for Met 16 in the mature protein. It is encoded 33 amino acids downstream of the initial methionine. This could provide an alternative translation initiation site. However, this would result in a loss of the signal peptide (first 17 amino acid residues) as well as additional 16 amino acids of the matured protein. Most likely, SNP 13 can only exist as heterozygote polymorphism, because a homozygote base exchange at this position would result in a lethal COX-2 knock-out.

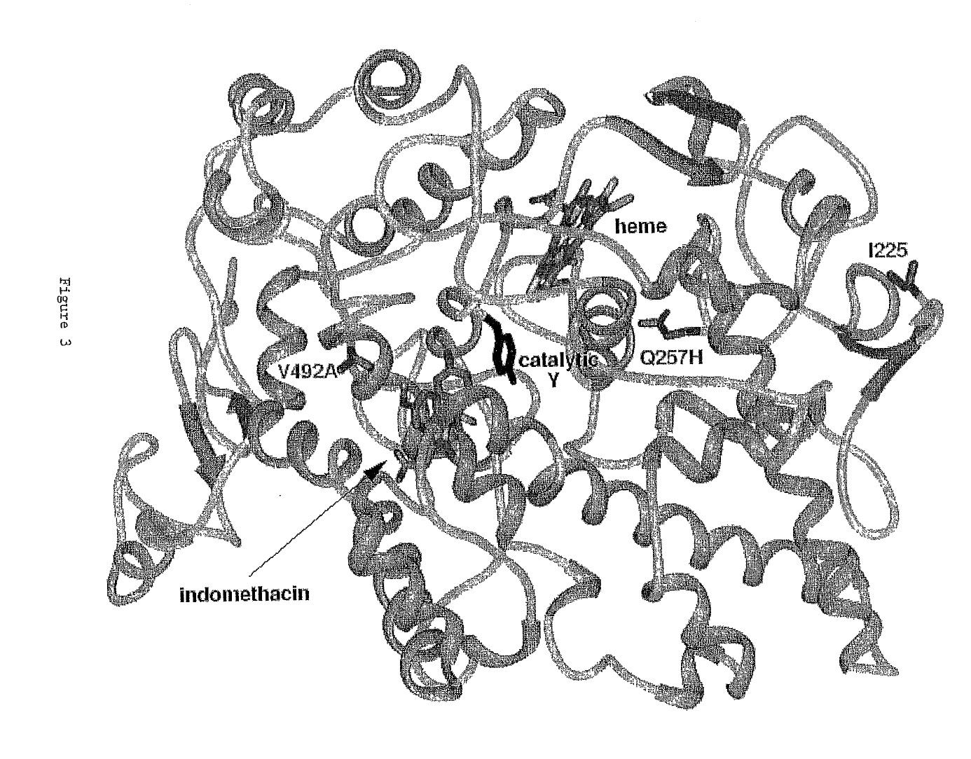

SNP 20 is located in exon 6. It represents a G to C transition at position 6055 and results in an amino acid exchange of glutamine 257 to histidine (mature protein). In the crystal structure of the mouse COX-2 protein (see FIG. 3) this position is located in a loop, which is 10 Å away from the heme prosthetic group. Although there are no intervening residues between Gln 257 and the heme group, the Gln to His mutation could dramatically affect the electrostatic potential via its interaction with solvent molecule. The notion that Gln 257 resides in a very conserved motif in many mammalian species in COX-2 as well as COX-1 protein sequences (see FIGS. 4A-4B) emphasizes an important role of that residue for the structure/function of the protein.

SNP 21 impacts the codon for histidine 384 by changing T 6721 to C located in exon 7. This changes a CAT to the alternative histidine-encoding CAC codon. The codon usage frequencies reported in the Kazusa database (http://www.kazusa.or.jp) for the two histidine codons in human genes are 41% and 59 %, respectively. Depending on the availability and relative abundance of the respective tRNA^{His} iso-acceptors, the change can affect the rate and fidelity of translation.

SNP 25 at position 8031 (exon 9) impacts protein sequence at position 492 of the mature protein, changing a valine to an alanine. In the crystal structure of the mouse COX-2 protein (FIG. 3), valine 492 is located in an alpha-helix in the active site groove. Val 492 corresponds to a Leu in the mouse protein and to an alanine in the sheep and in the bovine protein. This residue is just 2 amino acids located from valine 490 (i.e. valine 523 in the mouse crystal structure, H. M.Berman, J.Westbrook, Z.Feng, G.Gilliland, T. N.Bhat, H.Weissig, I. N.Shindyalov, P. E.Bourne, 2000.The Protein Data Bank. Nucleic Acids Research 28, 235-242), which plays an important role in discriminating cyclooxygenase-2 from cyclooxygenase-1 active site topography. As COX-1 shows an isoleucine at that position, Val 490 influences the topography as well as flexibility of the active site during binding of COX-inhibitors. Detailed knowledge of the active site structure was essential for the design of COX-2 specific inhibitors (Luong et. al. 1996; Kalgutkar et al. 1998; Kurumbail et al., 1996). It will be very interesting to investigate whether human COX-2 specific inhibitors will show the same affinity/activity against sheep and bovine COX-2 which both have an alanine at position 492. This amino acid polymorphism could impact the binding and thus the efficacy of COX inhibitors in individuals carrying SNP 25.

The disclosure of each of the publications cited in the specification is hereby incorporated by reference herein in its entirety.

Unless defined otherwise, all technical and scientific terms used herein have the same meaning as commonly understood by one of ordinary skill in the art to which this invention relates. Although the invention has been set forth in detail, one skilled in the art will recognize that numerous changes and modifications can be made, and that such changes and modifications may be made without departing from the spirit and scope of the invention.

We claim:

1. An isolated nucleic acid comprising at least 15 consecutive nucleotide bases including a polymorphic site selected from the group consisting of:
a.) an A→G substitution at nucleotide 188 of the COX-2 gene,
b.) an A→G substitution at nucleotide 502 of the COX-2 gene,
c.) an A→C substitution at nucleotide 716 of the COX-2 gene,
d.) an A→G substitution at nucleotide 953 of the COX-2 gene,
e.) an A→G substitution at nucleotide 1048 of the COX-2 gene,
f.) an A→G substitution at nucleotide 1092 of the COX-2 gene,
g.) a G→C substitution at nucleotide 1478 of the COX-2 gene,
h.) a T→C substitution at nucleotide 1636 of the COX-2 gene,
i.) a C→G substitution at nucleotide 2080 of the COX-2 gene,
j.) a T→C substitution at nucleotide 2124 of the COX-2 gene,
k.) a C→G substitution at nucleotide 2181 of the COX-2 gene,
l.) a T→G substitution at nucleotide 2252 of the COX-2 gene,
m.) a G→A substitution at nucleotide 2379 of the COX-2 gene,
n.) an A→C substitution at nucleotide 2435 of the COX-2 gene,
o.) a C→G substitution at nucleotide 3444 of the COX-2 gene,
p.) a G→C substitution at nucleotide 3602 of the COX-2 gene,
q.) a G→C substitution at nucleotide 3726 of the COX-2 gene,
r.) a T→C substitution at nucleotide 4190 of the COX-2 gene,
s.) a G→A substitution at nucleotide 5872 of the COX-2 gene,
t.) a G→C substitution at nucleotide 6055 of the COX-2 gene,
u.) a T→C substitution at nucleotide 6721 of the COX-2 gene,
v.) a C→T substitution at nucleotide 7167 of the COX-2 gene,
w.) a T→C substitution at nucleotide 7474 of the COX-2 gene,
x.) a G→T substitution at nucleotide 7491 of the COX-2 gene, and
y.) a T→C substitution at nucleotide 8031 of the COX-2 gene; or its complement.

2. An isolated nucleic acid of claim 1, which is DNA.

3. An isolated nucleic acid of claim 1, which is RNA.

4. An isolated allele specific primer capable of detecting a COX-2 polymorphic site of claim 1.

5. An isolated allele specific oligonucleotide probe capable of detecting a COX-2 polymorphic site of claim 1.

6. A diagnostic kit comprising an allele specific primer of claim 4 or allele specific oligonucleotide of claim 5.

7. An isolated polypeptide comprising at least 5 consecutive amino acid bases, one or more of which are encoded by the nucleotides at a polymorphic site of claim 1 or its complement.

8. An isolated polypeptide comprising at least 5 consecutive amino acid bases including a polymorphic site selected from the group consisting of:
a.) a Met→Ile substitution at amino acid position 1 of the COX-2 protein,
b.) a Gln→His substitution at amino acid position 257 of the COX-2 protein, and
c.) a Val→Ala substitution at amino acid position 511 of the COX-2 protein.

9. An antibody that binds specifically to a polypeptide of claim 7.

10. An antisense oligonucleotide comprising at least 5 nucleotide bases of a polymorphic site claim 1.

11. A method of detecting a nucleic acids of claim 1 comprising a method selected from the group consisting of: restriction-fragment-length-polymorphism detection based on allele-specific restriction-endonuclease cleavage, hybridization with allele-specific oligonucleotide probes, oligonucleotide arrays, allele-specific PCR, mismatch-repair detection (MRD), denaturing-gradient gel electrophoresis (DGGE), single-strand-conformation-polymorphism detection (SSCP), RNAase cleavage at mismatched base-pairs, chemical or cleavage of heteroduplex DNA, methods based on allele specific primer extension, genetic bit analysis (GBA), the oligonucleotide-ligation assay (OLA), the allele-specific ligation chain reaction (LCR), gap, radioactive and/or fluorescent DNA sequencing, and peptide nucleic acid (PNA) assays.

12. The method of claim 11 for predicting the clinical response to a therapeutic compound, or for determining the therapeutic dose of a compound in the treatment of a COX-2 mediated disease.

13. The method of claim 11 for assessing the predisposition of an individual to diseases mediated by COX-2.

14. A method of treating a human in need of a COX-2 drug wherein the method includes:
a.) detecting of a single nucleotide polymorphism in the COX-2 gene in the human, which detection comprises determining the sequence of the nucleic acid at one or more positions in Table 3;
b.) determining the status of the human by reference to polymorphism in the COX-2 gene; and
c.) administering an effective amount of a COX-2 drug.

15. A pharmaceutical pack comprising a COX-2 drug and instructions for administration of the drug to humans diagnostically tested for a polymorphic site at one or more of positions of Table 3.

16. A computer readable medium comprising at least one nucleic acid of claim 1.
